(19) United States
(12) Patent Application Publication
(10) Pub. No.: US 2012/0136273 A1
(43) Pub. Date: May 31, 2012

(54) APPARATUS AND METHOD FOR MONITORING AND ANALYZING BRAINWAVES

(75) Inventor: Bruce V. Michelson, JR., St. Louis, MO (US)

(73) Assignee: EPILEPSY SOLUTIONS, LLC, St. Louis, MO (US)

(21) Appl. No.: 13/276,847

(22) Filed: Oct. 19, 2011

Related U.S. Application Data
(60) Provisional application No. 61/417,672, filed on Nov. 29, 2010.

(51) Int. Cl. A61B 5/0476

(52) U.S. Cl. 600/544

(57) ABSTRACT

A neurological monitoring and analysis system comprising electrodes disposable on a patient's head to detect neurological activity, an electrode retention device for securely retaining the electrode(s) on the patient's head and a warning alert device for providing a warning signaling that a particular neurological activity incident has occurred within the patient's brain. Additionally, the system comprises a computer based processing system operable to execute a brainwave diagnostic program to receive the neurological activity data from the electrode(s), analyze the received neurological activity data to identify an occurrence of the particular neurological activity incident, and transmit an activation signal to the warning alert device such that the warning alert device produces an observable notification indicating that the particular neurological activity incident has occurred.

### CROSS-REFERENCE TO RELATED APPLICATIONS

This application claims the benefit of U.S. Provisional Application No. 61/417,672, filed on Nov. 29, 2010. The disclosure of the above application is incorporated herein by reference in its entirety.

### FIELD

The present disclosure relates to a system and method for monitoring and analyzing brainwaves, and in particular, quantifying and qualifying electrographic data for brainwaves of an epileptic person.

### BACKGROUND

Epilepsy is a common chronic neurological disorder characterized by recurrent unprovoked seizures. These seizures are transient signs and/or symptoms of abnormal, excessive or synchronous neuronal activity in the brain. Epilepsy is more likely to occur in young children, or people over the age of 65 years, however, it can occur at any time.

Epilepsy is usually treated with medication prescribed by a physician. Parents, caregivers and neurologists, however, provide the primary care for people with epilepsy. The mainstay of treatment of epilepsy is anticonvulsant medications. The goal for such medications is to prevent all seizures with minimal side effects, and the job of the physician is to aid the patient to find the best balance between the two during the prescribing of anticonvulsants. Beyond symptoms of the underlying diseases that can be a part of certain epilepsies, young children with epilepsy are at risk of death from sudden unexpected epileptic seizures.

For hospitalized patients, healthcare personnel use electrodes placed on the patient's scalp in order to monitor brainwave activity. These electrodes are termed “wet” as personnel use gel is applied to the interface between the electrode and the scalp. After applying the gel and electrodes, the healthcare personnel wrap the entirety of the patient's head with gauze to immobilize the electrodes during diagnostic reading of the electrodes. Placing and removing the gel along with wrapping and unwrapping the gauze are cumbersome, uncomfortable and time consuming activities. Removing the gel, in particular, requires thorough cleaning of the patient's head.

For at-home patients, especially children or older adults requiring supervision, known application procedures of placing electrodes are inconvenient and ineffective for data-gathering during overnight monitoring. Accordingly, for parents or caregivers at home, there are no current brainwave monitoring systems that are cost effective or provide easily quantifiable and qualifiable electrical readings for general wave patterns, epileptic form discharges or seizures that may occur during sleep.

Typically, to alert parents of an epileptic episode, at night parents place a motion sensing device on their child or in proximity to their child, e.g., on their child's bed. These motion devices sense the movement of the child and/or vibration of the bed to emit an alarm possibly indicating a night seizure. These devices can often provide false-positive alarms due to normal sleep movement leading to loss of sleep for parents and tiredness for the parents for actual alarms. Still further, these devices often miss non-moving seizures due to monitor capabilities. Even when the devices correctly alert the parents, these devices do not quantify or qualify pre-onset and post onset brainwave activities. Additionally, parents can video monitor their children to digitally record nighttime movement and associated seizures. Recorded video activity, however, is typically reviewed post-hoc, often several hours after a seizure actually occurred.

### SUMMARY

In various embodiments, the present disclosure provides a neurological monitoring and analysis system for monitoring and analyzing brainwaves of a person with a neurological disorder. Generally, the neurological monitoring and analysis system includes one or more electrodes disposable on a patient's head to detect neurological activity and an electrode retention device for securely retaining the electrode(s) on the patient's head. Additionally, the neurological monitoring and analysis system includes a warning alert device operable to provide a warning signaling indicating that a particular neurological activity incident has occurred within the patient's brain. The neurological monitoring and analysis system further includes computer based processing system operable to execute a brainwave diagnostic program to receive the neurological activity data from the electrode(s), analyze the received neurological activity data to identify an occurrence of the particular neurological activity incident, and transmit an activation signal to the warning alert device. Upon receipt of the activation signal the warning alert device produces a notification, observable by medical personnel, a caregiver or a parent of the patient, indicating that the particular neurological activity incident has occurred.

Further areas of applicability of the present teachings will become apparent from the description provided herein. It should be understood that the description and specific examples are intended for purposes of illustration only and are not intended to limit the scope of the present teaching.

### DRAWINGS

The drawings described herein are for illustration purposes only and are not intended to limit the scope of the present teachings in any way.

FIG. 1 is a schematic block diagram of a neurological monitoring and analysis system, in accordance with various embodiments of the present disclosure.

FIG. 2 is a schematic illustrating an electrode retention device of the neurological monitoring and analysis system shown in FIG. 1, in accordance with various embodiments of the present disclosure.

FIG. 3 is a flowchart illustrating a method of use for the neurological monitoring and analysis system shown in FIG. 1, in accordance with various embodiments of the present disclosure.

Corresponding reference numerals indicate corresponding parts throughout the several figures of the drawings.

### DESCRIPTION

The following description is merely exemplary in nature and is in no way intended to limit the present teachings, application, or uses. Throughout this specification, like reference numerals will be used to refer to like elements.

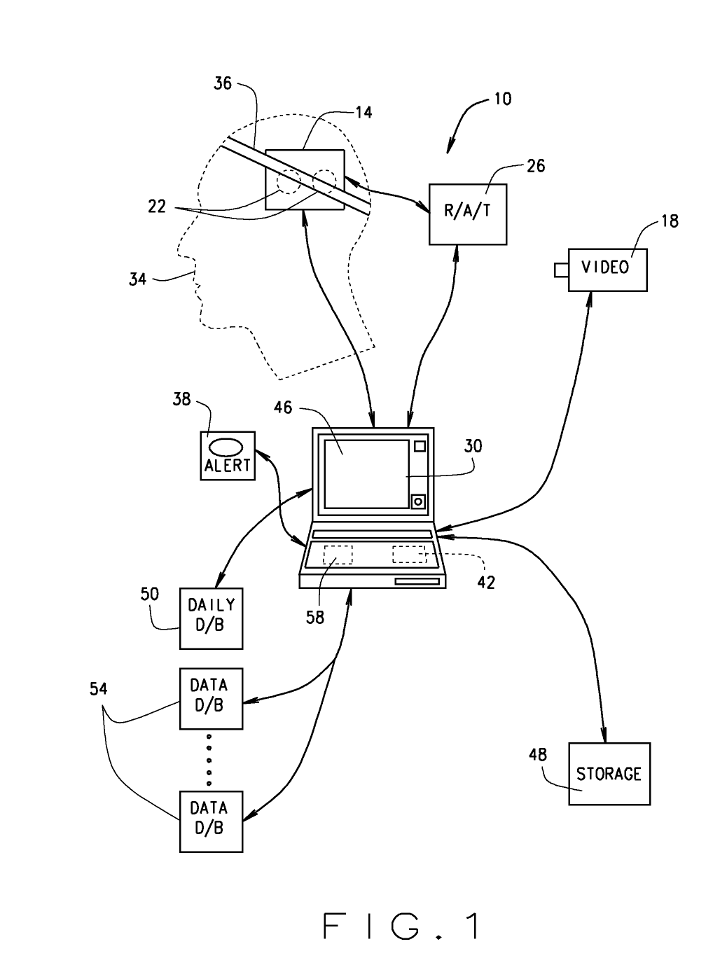

Referring to FIG. 1, the present disclosure provides a neurological monitoring and analysis system 10 for monitoring and analyzing neurological activity, i.e., brainwaves, in a person's brain, e.g., brainwaves of a person with a neurological disorder. The system 10 can be used for any neurological activity detection. However, for purposes of illustration only, the system 10 will be described herein as a system for monitoring and analyzing brainwaves of an epilepsy patient, particularly a child patient.

Generally, the system 10 comprises an electrode retention device 14, a video monitoring device 18, one or more electrodes 22, a signal receiver/amplifier/transmitter device 26 for transmitting, amplifying and receiving signals from the electrodes 22, a computer based processing system 30 operable to execute software for monitoring, acquiring and analyzing brainwaves of a patient, and an warning alert device 38 for providing a warning or alert of an impending or presently occurring epileptic seizure. In the illustrated embodiments, the system 10 is configured as a non-medical device. However, in various embodiments, the system 10 can be structured and operable to adhere to regulations such that it can be classified as a medical device.

The processing system 30 can be a local system (e.g., located in the same room as the patient) or a remote system (e.g., located at a different location than the patient). Additionally, communication links between the various components of the system 10 (i.e., the processing system 30, the electrode retention device 14, the video monitoring device 18, the electrodes 22, the receiver/amplifier/transmitter device 26 and the warning alert device 38) can be wired and/or wireless. Furthermore, the various components of the systems can be of any size and of any configuration to accommodate multiple characterizations of users and patients.

The electrode retention device 14 is structured and operable to be removably disposed on the patient's head 34 in a convenient manner to securely retain the electrodes 22 on the patient's head 34. Particularly, the electrodes 22 are removably attached to the patient's scalp in the manner, location and/or pattern as is commonly known for short term, e.g., fifteen to thirty minutes, monitoring of such neurological activity, i.e., brainwave monitoring. However, via the electrode retention device 14, the electrodes 22 can be held steadily in place for long term, e.g., one to 24 hours or more, monitoring. Specifically, the electrode retention device 14 steadily maintains the electrodes 22 in place on the patient's head 34, i.e., prevents movement of the electrodes 22 on the patient's head 34, for an extended period of time, e.g., 1, 3, 6, 8, 10, 12, 24 hours or more. In various embodiments, when the electrode retention device 14 is in place, it applies modest to measurable pressure on the electrodes 22 and the scalp to maintain electrodes 22 in place regardless of where the electrodes 22 are placed. This provides significant flexibility and speed for placing the electrodes 22 and maintaining them over short or extended periods of time.

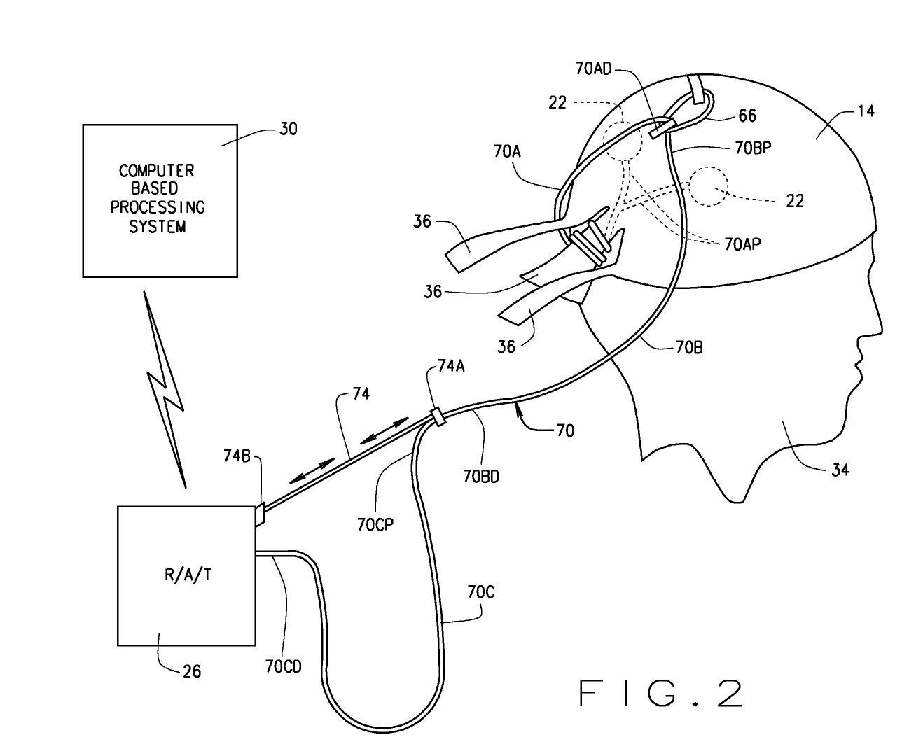

Referring now to FIGS. 1 and 2, in various embodiments, the electrode retention device 14 can comprise a one-piece substantially form-fitting head cover or swath of soft material, particularly, not a device that is repeated wrapped around the patient's head 34. For example, in various embodiments, the electrode retention device 14 can be a cloth head cover similar to what is sometimes referred to a Do Rag', or a head cover similar to a surgical cap. In such implementations, the head cover (referred to herein as head cover 14) can be a pre-shaped form-fitting cloth or fabric head cover that can be fitted on the patient's head 34. Or, alternatively, in other implementations, the head cover 14 can be a square or rectangular swath of fabric that can be placed on the patient's head 34 and folded about the patient's head 34 such the swath is substantially form-fitting to patient's head 34. It is envisioned that the head cover 14 can be constructed in various sizes and shapes for different ages and significantly different shaped heads. In such embodiments, the head cover 14 can include absorbent pads on an inner side to absorb fluids such as sweat emitted by the patient while wearing the head cover 14.

Generally, the electrode retention device 14 includes one or more fasteners 36 that are operable to snugly dispose and retain the electrode retention device 14 on the patient's head 34. For example, various exemplary forms, the fastener(s) 36 can comprise an elastic head band or strap that elastically fits around the patient's head 34 to securely retain the electrode retention device 14 on the patient's head 34. Or, in other exemplary forms, the fastener(s) 36 can comprise any other reusable, manually operated fastening device suitable for securely retaining the electrode retention device 14 on the patient's head 34, e.g., a string or ribbon that can be manually tied and untied around the patient's head 34 or any other device, apparatus or system suitable for securely maintaining the electrode retention device 14 on the patient's head 34.

For example, in the various implementations wherein the electrode retention device 14 is the head cover 14 exemplarily illustrated in FIG. 2, the fasteners 36 can comprise two or more tie tabs that can be tied at the back of the patient's head 34 to secure the head cover 14 firmly in place on patient's head 34 such that the electrodes 22 are securely retained on the patient's scalp. Or, in various implementations, the fasteners 36 can comprise the corners of an exemplary square or rectangular swath of fabric placed on the patient's head 34 that can be tied at the back of the patient's head 34 to secure the swath firmly in place on patient's head 34. Alternatively, in various forms, the fastener(s) 36 can comprises hook-and-loop interlocking tabs or adhesive tabs that extend from a rear of the electrode retention device 14, e.g., the head cover 14, to securely retain the electrode retention device 14 on the patient's head 34 in a hat-like manner.

With further reference to FIG. 2, wherein the electrode retention device 14 is exemplarily illustrated as a form-fitting head cover 14 having a plurality of tie tabs 36 (i.e., fasteners 36) extending from a back side of the head cover 14, the head cover 14 can include an accessory loop 62 disposed on a top portion of the head cover 14. The accessory loop 62 can be sewn or otherwise affixed to the head cover 14, or integrally formed with the head cover 14. The accessory loop 62 is structured to allow a detachable tension relief ring 66 to be removably connected to the top of the head cover 14. The tension relief ring 66 is structured and operable to have one or more electrode wire leads 70 connected thereto, such that the wire lead(s) 70 are removably connected to the top of the head cover 14. More specifically, in various embodiments, the electrodes 22 are communicatively connected to the receiver/amplifier/transmitter device 26 via the one or more wire leads 70 and the receiver/amplifier/transmitter device 26 is wirelessly communicatively connected to the computer based processing system 70.

Accordingly, the receiver/amplifier/transmitter device 26 can be a mobile unit located near the patient or attached to the patient, via a backpack, holster or other suitable device, such that the patient can have the ability to move about without being directly connected to the processing system 70. In such embodiments, the electrode lead(s) 70 is/are described as having three sections; a fixed section 70A, at tether section 70B and a slack section 70C. The electrode lead fixed section(s) 70A include(s) a proximal end 70AP that is connected to the electrodes 20 and a distal end 70AD that is fixedly connected to the tension relief ring 66. Additionally, the electrode lead tether section(s) 70B include(s) a proximal end 70BP is that is fixedly connected to the tension relief ring 66 and a distal end 70BD that is fixedly connected to a first end 74A of a slack elimination device 74. A second end 74B of the slack elimination device 74 is fixedly connected to the receiver/amplifier/transmitter device 26 or an apparatus housing the receiver/amplifier/transmitter device 26, e.g., a backpack or a holster. Furthermore, the slack section(s) 70C of the electrode lead(s) 70 include(s) a proximal end 70CP fixedly connected to the slack elimination device first end 74A and a distal end 70CD that is fixedly connected to the receiver/amplifier/transmitter device 26 or an apparatus housing the receiver/amplifier/transmitter device 26, e.g., a backpack or a holster.

Due to the fixed section distal end 70AD and the tether section proximal end 70BP being fixedly connected to the tension relief ring 66 any stress, i.e., tugging or pulling, on the tether section 70B of the electrode lead(s) 70 resulting from movement of the patient or of the receiver/amplifier/transmitter device 26 will not be translated to the fixed section 70A of the electrode lead(s) 70. Rather, such stress, i.e., tugging or pulling, on the tether section 70B of the electrode lead(s) 70 such stress, i.e., tugging or pulling, on the tether section 70B of the electrode lead(s) 70 will be translated and absorbed by the head cover 14 via the fixed connection of the tether section proximal end 70BP to the tension relief ring 66. Importantly, because of the fixed connection of the tether section proximal end 70BP to the tension relief ring 66 such stress, i.e., tugging or pulling, on the tether section 70B of the electrode lead(s) 70 will be substantially terminated at the tension relief ring and not be translated to the electrode lead(s) fixed section(s) 70A and the electrodes 22. Therefore, the electrodes 22 will not be dislodged, moved or disturbed from their location/position on the patient's head 14 if the patient moves or the receiver/amplifier/transmitter device 26 is moved causing stress, i.e., tugging or pulling, on the tether section 70B of the electrode lead(s) 70.

In various implementations, to further insure that the electrodes 22 will not be dislodged, moved or disturbed from their location/position on the patient's head 14 if the patient moves or the receiver/amplifier/transmitter device 26 is moved causing stress, i.e., tugging or pulling, on the tether section(s) 70B of the electrode lead(s) 70, the fixed section(s) 70A can be secured at the back of the head cover 14 using the fasteners 36, i.e., the tie tabs 36. More specifically, the electrode lead fixed section(s) 70A can be wrapped around one or more of the tie tabs 36 prior to tying the tie tabs 36 together to secure the head cover 14 in place on the patient's head 34. Subsequently, tying the tie tabs 36 together will secure the head cover 14 in place on the patient's head 34 and secure a portion of the fixed section(s) 70A within the resulting knot. This will provide further strain relief if the tether section(s) 70B is/are tugged or pulled due to movement of the patient or the receiver/amplifier/transmitter device 26.

As described above, in various implementations, a slack elimination device 74 is connected at a first end 74A to the proximal end(s) 70CP of the electrode lead slack section(s) 70C and at a second end 74B to the receiver/amplifier/transmitter device 26 or an apparatus housing the receiver/amplifier/transmitter device 26, e.g., a backpack or a holster. The slack elimination device 74 can be any device, apparatus or mechanism structured and operable to eliminate excessive slack in the tether section(s) 70B of the electrode lead(s) 70.

Specifically, the slack elimination device 74 is structured and operable to apply a slight tension between the proximal end(s) 70CP and the distal end(s) 70CD of the electrode lead(s) slack section(s) 70C. The tension between the slack section(s) proximal end(s) 70CP and the distal end(s) 70CD causes any excessive slack between the tether section(s) proximal end(s) 70BP and the slack section(s) distal end(s) 70CD to be localized within the slack section(s) 70C, i.e., between the proximal and distal ends 70CP and 70CD of the slack section(s) 70C. More particularly, by localizing any slack within the slack section(s) 70C, excessive slack is prevented from occurring in the tether section(s) 70B, thereby keeping an excessive amount of the electrode lead(s) 70 from accumulating near the patient's head 34 and/or neck. Rather, any slack in the electrode lead(s) 70C between the head cover 14 and the receiver/amplifier/transmitter device 26, is caused to accumulate within the slack section(s) 70C, i.e., between the between the slack section(s) proximal end(s) 70CP and the distal end(s) 70CD, away from the patient's head 34 and/or neck.

As described above, the slack elimination device 74 can be any device, apparatus or mechanism structured and operable to eliminate excessive slack in the tether section(s) 70B of the electrode lead(s) 70. For example, in various embodiments, the slack elimination device 74 can be an elastic strap that will stretch when the tether section(s) proximal end(s) 70CP are pulled away from the tether section(s) distal end(s) 70CD due movement of the patient away from the receiver/amplifier/transmitter device 26, or vice versa. Subsequently, if the patient moves closer to the receiver/amplifier/transmitter device 26, or vice versa, elastic strap will retract causing slack to gather between the slack section(s) proximal end(s) 70CP and the distal end(s) 70CD.

Similarly, in various other embodiments, the slack elimination device 74 can be a rubber band, a light gauge spring, or recoiling string or wire attached to a biased pulley or spool, whereby the rubber band, spring or recoiling string/wire will stretch/extend when the tether section(s) proximal end(s) 70CP are pulled away from the tether section(s) distal end(s) 70CD due movement of the patient away from the receiver/amplifier/transmitter device 26, or vice versa. And, subsequently, if the patient moves closer to the receiver/amplifier/transmitter device 26, or vice versa, rubber band, spring or recoiling string/wire will retract/recoil causing slack to gather between the slack section(s) proximal end(s) 70CP and the distal end(s) 70CD.

The electrodes 22 operatively connect, either through a wired or wireless connection, to the receiver/amplifier/transmitter device 26. The electrodes 22 monitor and acquire neurological activity data, i.e., brainwave data, indicative of the patient's brain activity and communicate the data, via a data signal, to the receiver/amplifier/transmitter device 26. Subsequently, the receiver/amplifier/transmitter device 26 conditions the received data, e.g., smooth's the data signal, converts the data signal from analog data to digital date, etc., and transmits, either through a wired connection or wirelessly, the brainwave data to the computer based processing system 30. Via execution of a brainwave diagnostics software program or algorithm 42 (hereafter brainwave diagnostics software program 42), e.g., an epileptic seizure diagnostics software program, executed by the computer based processing system 30, the computer based processing system 30 compiles and analyzes the brainwave data.

One skilled in the art will readily understand that a software program, e.g., the brainwave diagnostics software program 42, does not directly receive and output data, rather it is the receipt of data by a computer based processing system, e.g., the computer based processing system 30, and the execution of the software program by a processor 58 of the computer based processing system 30, utilizing the received data, that produces an output. However, for simplicity, inputs and outputs will be referred to herein with reference only to the brainwave diagnostics software program 42. That is, for simplicity, references will be made to the brainwave diagnostics software program 42 receiving data and producing outputs based on the received data, without further description of the execution of the brainwave diagnostics software program 42 by the processor 58 the computer based processing system 30, to produce the outputs described herein.

In various embodiments, after the brainwave diagnostics software program 42 analyzes the receive brainwave data and outputs the resulting analysis data to an output device 46, e.g., a computer display device, where the analysis data can be viewed by medical personnel, a caregiver or a parent of the patient. In other embodiments, the resulting analysis data can be electronically stored for future review by medical personnel, a caregiver or a parent.

Furthermore, in various implementations, during the electrode monitoring, the video monitoring device 18 can record the patient and transmit video data of the patient's movements to a video display (locally or remotely located) or for storage on an electronic storage device 48 (located locally on the computer based processing system 30 or remotely therefrom) for future review by medical personnel or a parent. Moreover, the video data can be analyzed by the brainwave diagnostic program 42 to identify the occurrence of a particular physical activity incident, e.g., a physical seizure. In various embodiments, the stored video data can be utilized to correlated patient activity and movements with particular neurological activity incidents, e.g., with seizure occurrence or onset. Additionally, in various implementations, via such correlations, the video data can be utilized by the brainwave diagnostic program 42 to identify the occurrence of the particular neurological activity incidents, e.g., seizure occurrence or onset.

Particularly, in various embodiments, the brainwave diagnostics software program 42 analyzes electroencephalogram (EEG) waveforms received from the receiver/amplifier/transmitter device 26. In common usage, the term “EEG” is used to refer to signals representing aggregate neuronal activity potentials detectable via the electrodes 22. In another embodiment (not shown), the term can also refer to signals obtained from deep in the patient's brain via depth electrodes and the like.

Generally, the system 10 is structured and operable to monitor a patient's neurological activity, analyze the patient's neurological activity and indicate when a particular incident of neurological activity, e.g., one or more particular neurological activities or brainwave patterns, has occurred within the patient's brain. For example, it is envisioned that the system 10, as described herein, will be utilized to monitor brain activity of an epileptic patient to detect, identify and indicate that a particular previously identified incident of brain activity, e.g., a particular previously identified pattern of brainwaves, has occurred that is indicative of the possible occurrence of an epileptic seizure. Therefore, if possible, the seizure can be treated prior to, at or near the beginning of the seizure. It is further envisioned that the system 10 can utilized to quantify and qualify at least one EEG signal data within at least one parameter over a significant amount of time, e.g., throughout the night while the patient is sleeping. More specifically, in various embodiments, the brainwave diagnostic program 42 analyzes the received brainwave data signals, e.g., the EEG data, to identify the occurrence of one or more particular neurological activity incidents, i.e., brain activities or brainwave patterns, e.g., one or more particular patterns of neuronal activity in one or more particular parts of the patient's brain that are indicative of a subsequent or present epileptic seizure. Then, if such brain activity is identified, the brainwave diagnostic program 42 outputs an activation signal to the warning alert device 38, whereafter the warning alert device 38 produces, generates or emits an observable notification, e.g., an alarm or warning such as a sound or light, notifying medical personnel, a caregiver or a parent of the patient of occurrence of the one or more particular brain activities.

For example, in various implementations, the brainwave diagnostic program 42 can analyze EEG data in a plurality of ways such as, but not limited to: (a) analyze at least one received EEG signal; (b) analyze any individual or range of frequencies within the at least one receive EEG signal; or (c) analyzed relationships between the amplitude and/or frequencies of the at least one receive EEG signal and empirically acquired statistical data of previously analyzed EEG signals. Moreover, the brainwave diagnostic program 42 quantifies and qualifies the gathered EEG data to assist the parents, caregivers, or medical personnel in teaching or “coaching” the parent, caregiver, or medical personnel to associate daily activities with abnormal brainwave activity and resultant seizures. Therefore, it is possible that undesirable results of such brainwave activity, such as an epileptic seizure, can be detected prior to or substantially simultaneously with onset, and treated/responded to prior to or substantially immediately after onset.

For example, the gathered EEG data can be compared to a daily stimuli database 50 comprising data regarding external stimuli experienced by the patient, e.g., parameters such as physical activities, food consumption, different light sources, various sounds, during that particular day. Or, the EEG data from the patient can be compared to data stored in one or more empirical data databases 54 comprising EEG data and comparisons to various parameters of a plurality of other patients. Accordingly, the brainwave diagnostic program 42 can analyze such daily information and compare the information to any particular neurological activity incident, e.g., abnormal brainwave activity or undesirable results of such brainwave activity, e.g., an epileptic seizure, which may subsequently occur such that such correlations can be tracked and used to prevent or quickly treat future undesirable results of such brainwave activity e.g., an epileptic seizure. Additionally, statistical analysis can be performed to correlate any association among the daily log parameters. Accordingly, parents, caregivers, or medical personnel can structure or manage care decisions throughout the day based on the compiled statistical analysis.

For example, with reference to epileptic seizures, the beginning of a seizure is referred to herein as an “onset.” However, it is important to note that there are two general varieties of seizure onsets. A “clinical onset” represents the beginning of a seizure as manifested through observable clinical symptoms, such as involuntary muscle movements or neurophysiologic effects such as lack of responsiveness. An “electrographic onset” refers to the beginning of detectable electrographic activity indicative of a seizure. An electrographic onset will frequently occur before the corresponding clinical onset, enabling intervention before the patient suffers symptoms, but that is not always the case. In addition, there are changes in the EEG that occur seconds or even minutes before the electrographic onset that can be identified, and may be used to facilitate intervention before clear electrographic or clinical onsets occur. This capability would be considered seizure anticipation, in contrast to the detection of a seizure or its onset.

Thus, the system 10 uses EEG monitoring to provide “real time” and continuous information concerning brain function of the patient during any desired period of time, e.g., through the night. In response, the system 10 generates an alarm/warning to alert the parent, caregiver, or medical personnel of undesirable brainwave activity that may be indicative of an oncoming undesirable result, e.g., an epileptic seizure. Additionally, in the instance of epileptic seizure analysis, for nighttime monitoring, the system reduces or eliminates false-positive alarms by providing alarms based on EEG analysis as opposed to actual motions by the patient.

Moreover, the compiled EEG data can be used to identify changes in brain function and provide prognostic markers, which can be useful to monitor response to therapy. Particularly, for epileptic seizure analysis, specific EEG measures can provide relevant information about brain function prior to seizure manifestation, which presents an opportunity for appropriate interventions by the parent, caregiver, or medical personnel. Additionally, capturing a seizure on video by the camera of the system 10 that is closely correlated with EEG seizure activity provides an enhanced based diagnosis of a seizure of epileptic origin.

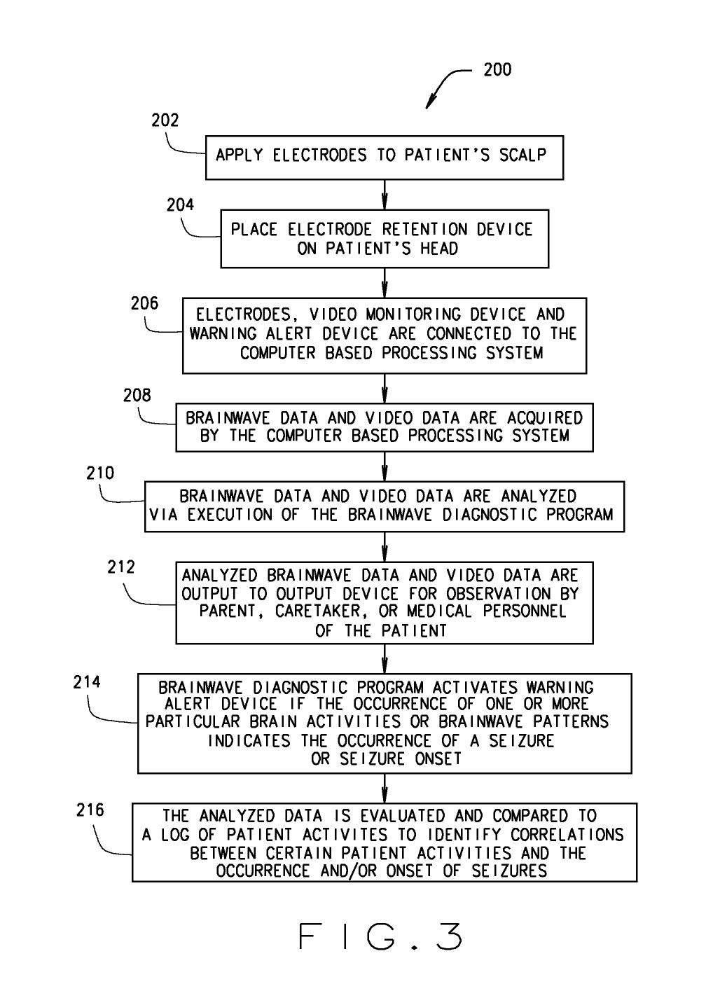

Referring now to FIG. 3, with further reference to epileptic seizure analysis the operation of the system 10, in an exemplary embodiment, will now be described referring to flow chart 200. As described above, in various embodiments, the system 10 is utilized to monitor the patient's brainwaves, via the electrodes 22 and receiver/amplifier/transmitter device 26, and the patient's movement, via the video monitoring device 18. Initially, the electrodes 22 are disposed on the patient's scalp in selected locations to monitor corresponding areas of the patient's brain, as indicated at 202. Next, the electrode retention device 14 is placed on the patient's head 34 to securely retain the electrodes 22 at the selected locations of the patient's scalp, as indicated at 204. Subsequently, the electrodes 22 are connected to the computer based processing system 30, via the receiver/amplifier/transmitter device 26, the video monitoring device 14 is aimed at the patient and connected to computer based processing system 30, and the warning alert device 38 is connected to the computer based processing system 30, as indicated at 206. As described above, all such connection can be made via a wired and/or wireless connection.

Next, the brainwave data and video data are captured/acquired by the computer based processing system 30, as indicated at 208. The acquired data is then evaluated by the brainwave diagnostic program 42 executed by the computer based processing system 30, as indicated at 210. In various implementations, graphical representations of the analyzed brainwave, e.g., a wave index, and/or the video data received from the video monitoring device 18 can be output to the output device 46, e.g., a video display monitor, where the data can be viewed by parent, caregiver, or medical personnel of the patient, as indicated at 212.

Subsequently, if the brainwave diagnostic program 42 identifies the occurrence of one or more particular neurological activity incidents, i.e., one or more particular brain activities or brainwave patterns, e.g., one or more particular patterns of neuronal activity in one or more particular parts of the patient's brain that are indicative of a subsequent or present epileptic seizure, the brainwave diagnostic program 42 transmits an activation signal to the warning alert device 38 to provoke an alarm warning the parent, caregiver, or medical personnel of such brain activities, as indicated at 214. The alarm can be visual (e.g. displayed on the output device 46), aural, or any other sort, variety, form or combination of signal(s) that convey information. In various implementations, a specific type of alarm may be associated with the identification of a specific type of brainwave activity identified to be indicative a particular type of seizure, thereby assisting the parent, caregiver, or medical personnel in response and appropriate medication.

In various implementations, the brainwave diagnostic program 42 further evaluates and compares the analyzed data with the daily log database 50 of patient activities and identifies correlations between certain patient activities and the occurrence and/or onset of seizures, as indicated at 216. Accordingly, these identified correlations between seizures and patient activities can be utilized by the parent, caregiver, or medical personnel of the patient to avoid such activities.

Referring now to FIGS. 1 and 2, the system 10 can be utilized to capture the patient's EEG, process the EEG using various measures, and then compare the processed results against baseline values or thresholds with respect to historical data of normal and abnormal brain function or behavior of the patient accumulated over time and stored in the electronic storage device 48. Based on the comparison, detection or identification of seizures or other abnormal brain function can be performed on a real time basis, and the alarm or alert can be provided to the parent, caregiver, or medical personnel in the event that abnormalities requiring attention are detected.

For example, in various implementations, the brainwave diagnostic program 42 compares all the output brainwave data with individual pre-established optimal normal brainwave ranges to analyze the patient's present, real time brainwave activity. That is, in various implementations, the brainwave diagnostic program 42 can compare the presently monitored brainwave frequencies, combination of frequencies, quantities of frequencies, types of brainwaves, e.g., delta wave or theta waves (i.e., theta rhythms), to those of the patient's normal brain activity (stored in the storage device 48) to identify the onset of a seizure.

Furthermore, in various implementations, the different measures or comparisons of the different measures with corresponding normal ranges can be combined into a single composite index value for the patient. If the index value is above or below a predetermined threshold value, the brainwave diagnostic program 42 triggers the alarm to alert the parent, caregiver, or medical personnel of a seizure or the possibility of the onset of a seizure. Still further, in various implementations, the brainwave diagnostic program 42 can detect seizures and seizure onsets using a method weighting function that evaluates all the linear and non-linear measures to the patient's EEG data. In various embodiments, detection of a seizure or seizure onset is declared if a particular parameter of the brainwave(s) monitored by any electrodes 22 is above or below preset threshold values and/or differ significantly from predetermined baseline values.

Additionally, in various other implementations, if there are certain measured brainwave parameter or combinations of measured brainwave parameters that indicate seizure onset (or indicated likelihood of seizure onset) at the same time, the brainwave diagnostic program 42 concludes that a seizure or seizure onset has occurred. Moreover, in various embodiments, a plurality of triggers can be utilized for detecting a seizure or possible seizure onset. For example, a trigger may comprise monitoring and analyzing delta wave patterns and/or theta wave, i.e., theta rhythm, patterns occurring repeatedly within a range of average amplitudes in conjunction with EEG signal data. Another example of an envisioned trigger is simply monitoring and analyzing EEG data, and/or monitoring and analyzing a combination of delta waves, theta waves/rhythyms and EEG data occurring repeatedly while the standard deviation of EEG data is below a certain amount. These triggers can be variable depending upon the patient and type of seizures.

It is envisioned that the computer based processing system 30 of the system 10, as described herein, can be a general-purpose computer and the brainwave diagnostic program or algorithm 42 can be implemented as software executed by the processor 58 of the computer based processing system 30. Such implementation provides a convenient user interface and allows easy selection, modification and/or updating of the brainwave diagnostic program or algorithm 42. Alternatively, it is envisioned that the computer based processing system 30 of the system 10, as described herein, can be any other computer based system or device such as a smart phone, hand held computer or other computer based system/device that comprises a processor capable of executing the brainwave diagnostic program or algorithm 42.

Further, the warning alert device 38 can be any device suitable for alerting the parent, patient or caregiver that a seizure or possible seizure onset has occurred. For example, in various embodiments, the warning alert device 38 can be (but is not limited to) an audible alarm and/or visible alarm, e.g., an illuminating alarm, that receive an alarm signal initiated by the brainwave diagnostic program 42 and transmitted by the computer based processing system 30, via a wired connection, a wireless connection or an Internet connection. Or, the warning alert device 38 can generate a progressive alarm which gradually provides a more intense stimulus and/or changes the kind of stimulus (e.g., from a music broadcast to a buzzer) to the parent, caregiver, or medical personnel.

It is envisioned that the system 10, as described herein, can be utilized by parents, caregivers, and medical personnel to provide, inter alia, a seizure alarm for epileptics, an epilepsy coach to assist epileptics associate daily activities with seizures and abnormal brainwaves, a system for holding electrodes to the patient's scalp, a method of local monitoring of live brainwave activity, a method of remote monitoring of live brainwave activity, and a method of reporting brainwave activity. The system 10 evaluates the patient's EEG data and triggers an alarm if a seizure or seizure onset is detected, and also acquires, analyzes, accumulates and records brainwave data during a seizure to help identify particular activities that may trigger or cause a seizure.

In an embodiment (not shown), the system 10 can also monitor, acquire and analyze other physical stimuli such as, but not limited to, temperature (e.g. from a nasal thermometer), heart rate, oxygen saturation (e.g. pulse oximetry), electromyogram (EMG) data, electrocardiogram (EKG), level of consciousness, and so forth. Other data regarding the patient's condition (pulse oximetry readings, pulse rate, etc.) can be evaluated in conjunction with the EEG data to provide a more accurate evaluation of the patient's brain function and/or provide information that can be used in an automated fashion or by a health care professional to determine causation of abnormal brain function and thus aid in diagnosis or therapy. Furthermore, it is envisioned that the system 10 can be integrated into an overall automated monitoring system that tracks, evaluates, and reports on all important or critical aspects of the patient's health or physical condition.

Hence, the system 10, as described herein, provides the ability to identify brainwave activities in the acute setting and in real time through quantitative and qualitative means provide a greater degree of control over this disorder so that parents, physicians and clinicians can make more efficient and effective treatment and management decisions.

Those skilled in the art will recognize that the computer based process system 30 and the receiver/amplifier/transmitter device 26 can be consolidated into one machine/device or location. For example, a single general-purpose computer can be utilized to acquire/receive the brainwave data, process/condition the brainwave data, execute the brainwave diagnostic program 42 to analyzed the brainwave data, store the brainwave data and analysis thereof, and initiate alarm signals.

Still further, one skilled in the art will recognize that the computer based process system 30 and/or the receiver/amplifier/transmitter device 26 can be located offsite in a facility separate from where the patient is located such as a third party monitoring center that can evaluate the EEG data and send the appropriate alarms. Moreover, in such offsite embodiments, the system 10 can confidentially gather EEG data from multiple patients and analyze the multiple EEG data for a larger statistical analysis, e.g., analysis of epileptic seizure occurrence, and larger compilation of other parameters such as the daily log activities for research purposes.

The description herein is merely exemplary in nature and, thus, variations that do not depart from the gist of that which is described are intended to be within the scope of the teachings. Such variations are not to be regarded as a departure from the spirit and scope of the teachings.

What is claimed is:

1. A neurological monitoring and analysis system for monitoring and analyzing brainwaves of a person with a neurological disorder, said system comprising:
one or more electrodes structured and operable to be disposed on a patient's head and detect neurological activity within patient's brain;
an electrode retention device structured and operable to securely retain the electrode(s) on the patient's head, the electrode retention device including a detachable tension relief ring structured to have one or more electrode wire leads connected thereto, such that the wire lead(s) are removably connected to the electrode retention device, the tension relief ring operable to prevent tension on the wire lead(s) from causing the electrode(s) from moving on the patient's head;
a warning alert device structured and operable to provide a warning or alert signaling that the neurological activity detected by the electrodes indicates that a particular incident of neurological activity has occurred within the patient's brain; and
a computer based processing system comprising a processor and a brainwave diagnostic program executable by the processor, the computer based processing system structured and operable, via execution of the brainwave diagnostic program, to receive the neurological activity data from the electrode(s), analyze the received neurological activity data to identify an occurrence of the particular neurological activity incident within the patient's brain, and upon occurrence of the particular neurological activity incident transmit an activation signal to the warning alert device such that the warning alert device produces an observable notification indicating that the particular neurological activity incident has occurred.

2. The system of claim 1 further comprising a video monitoring device structured and operable to transmit video data of the patient to the computer based processing system for use by the brainwave diagnostic program to identify the occurrence of a particular physical activity incident.

3. The system of claim 1 further comprising a receiver/amplifier/transmitter device structured and operable to receive the neurological activity data from the electrode(s), condition the received neurological activity data, and transmit the conditioned neurological activity data to the computer based processing system.

4. The system of claim 1 further comprising a daily stimuli database comprising data regarding external stimuli experienced by the patient during a particular time period, the data from the daily log database accessible by the brainwave diagnostic program to develop correlations between particular external stimuli and the occurrence of the particular neurological activity incident of the patient.

5. The system of claim 1 further comprising an empirical data database comprising accumulated empirical data regarding neurological activity and the occurrence of the particular neurological activity incident of a plurality of subjects other than the patient, the data from the empirical data database accessible by the brainwave diagnostic program for use in identifying the occurrence of the particular neurological activity incident within the patient's brain.

6. The system of claim 1, wherein the electrode retention device comprises a one-piece substantially form-fitting head cover.

7. The system of claim 1 further comprising a slack elimination device structured and operable to eliminate excessive slack in a portion of the electrode wire lead(s) extending between the tension relief ring and a receiver/amplifier/transmitter device of the system that is structured and operable to receive the neurological activity data from the electrode(s), condition the received neurological activity data, and transmit the conditioned neurological activity data to the computer based processing system.

8. A neurological monitoring and analysis system for monitoring and analyzing brainwaves of a person with a neurological disorder, said system comprising:
one or more electrodes structured and operable to be disposed on a patient's head and detect neurological activity within patient's brain;
an electrode retention device structured and operable to securely retain the electrode(s) on the patient's head, the electrode retention device including a detachable tension relief ring structured to have one or more electrode wire leads connected thereto, such that the wire lead(s) are removably connected to the electrode retention device, the tension relief ring operable to prevent tension on the wire lead(s) from causing the electrode(s) from moving on the patient's head;
a warning alert device structured and operable to provide a warning or alert signaling that the neurological activity detected by the electrodes indicates that a particular incident of neurological activity has occurred within the patient's brain;
a computer based processing system comprising a processor and a brainwave diagnostic program executable by the processor, the computer based processing system structured and operable, via execution of the brainwave diagnostic program, to receive the neurological activity data from the electrode(s), analyze the received neurological activity data to identify an occurrence of the particular neurological activity incident within the patient's brain, and upon occurrence of the particular neurological activity incident transmit an activation signal to the warning alert device such that the warning alert device produces an observable notification indicating that the particular neurological activity incident has occurred; and
a receiver/amplifier/transmitter device structured and operable to receive the neurological activity data from the electrode(s), condition the received neurological activity data, and transmit the conditioned neurological data to the computer based processing system.

9. The system of claim 8 further comprising a video monitoring device structured and operable to transmit video data of the patient to the computer based processing system for use by the brainwave diagnostic program to identify the occurrence of a particular physical activity incident.

10. The system of claim 8 further comprising a daily stimuli database comprising data regarding external stimuli experienced by the patient during a particular time period, the data from the daily log database accessible by the brainwave diagnostic program to develop correlations between particular external stimuli and the occurrence of the particular neurological activity incident of the patient.

11. The system of claim 8 further comprising an empirical data database comprising accumulated empirical data regarding neurological activity and the occurrence of the particular neurological activity incident of a plurality of subjects other than the patient, the data from the empirical data database accessible by the brainwave diagnostic program for use in identifying the occurrence of the particular neurological activity incident within the patient's brain.

12. The system of claim 8, wherein the electrode retention device comprises a one-piece substantially form-fitting head cover.

13. The system of claim 8 further comprising a slack elimination device structured and operable to eliminate excessive slack in a portion of the electrode wire lead(s) extending between the tension relief ring and a receiver/amplifier/transmitter device of the system that is structured and operable to receive the neurological activity data from the electrode(s), condition the received neurological activity data, and transmit the conditioned neurological activity data to the computer based processing system.

14. A method for monitoring and analyzing brainwaves of a person with a neurological disorder, said method comprising:
disposing one or more electrodes, of a neurological monitoring and analysis system, on a patient's head to detect neurological activity within a patient's brain;
securely retaining the electrode(s) on the patient's head utilizing an electrode retention device of the neurological monitoring and analysis system;
connecting one or more electrode wire leads extending from the electrode(s) to a detachable tension relief ring of the neurological monitoring and analysis system, the tension relief ring removably connected to the electrode retention device such that tension on the wire lead(s) is prevented from causing the electrode(s) from moving on the patient's head;
receiving the neurological activity data from the electrode(s) at a computer based processing device of the neurological monitoring and analysis system, the computer based processing device comprising:
a processor; and
a brainwave diagnostic program executable by the processor;
analyzing the received neurological activity data, via execution of the brainwave diagnostic program, to identify an occurrence of the particular neurological activity incident within the patient's brain; and
transmitting an activation signal to a warning alert device of the neurological monitoring and analysis system, whereby the warning alert device provides a warning or alert signaling that the particular neurological activity incident has occurred within the patient's brain.

15. The method of claim 14 further comprising transmitting video data of the patient to the computer based processing system, via a video monitoring device of the neurological monitoring and analysis system, for use by the brainwave diagnostic program to identify the occurrence of a particular physical activity incident.

16. The method of claim 14 further comprising:
receive the neurological activity data from the electrode(s) at a receiver/amplifier/transmitter device of the neurological monitoring and analysis system;
conditioning the received neurological activity data via the receiver/amplifier/transmitter device; and
transmitting the conditioned neurological activity data from the receiver/amplifier/transmitter device to the computer based processing system.

17. The method of claim 14 further comprising develop correlations between particular external stimuli and the occurrence of the particular neurological activity incident of the patient utilizing data, accessible by the brainwave diagnostic program, regarding external stimuli experienced by the patient during a particular time period stored in a daily stimuli database of the neurological monitoring and analysis system.

18. The method of claim 14 further comprising identifying the occurrence of the particular neurological activity incident within the patient's brain utilizing data, accessible by the brainwave diagnostic program, regarding neurological activity and the occurrence of the particular neurological activity incident of a plurality of subjects other than the patient stored in an empirical data database of the neurological monitoring and analysis system.

19. The method of claim 14, wherein securely retaining the electrode(s) on the patient's head utilizing an electrode retention device comprises placing a one-piece substantially form-fitting head cover over the electrode(s) and securing the head cover in place utilizing fasteners of the head cover.

20. The method of claim 14 further comprising connecting a first end of slack elimination device of the neurological monitoring and analysis system to a portion of the wire lead(s) and a second end of the slack elimination device to the receiver/amplifier/transmitter device such that the slack elimination device eliminates excessive slack in a portion of the electrode wire lead(s) extending between the tension relief ring and a receiver/amplifier/transmitter device.
